(19) United States
(12) Patent Application Publication
(10) Pub. No.: US 2022/0288082 A1
(43) Pub. Date: Sep. 15, 2022

(54) TREATMENT OF DEMENTIA

(71) Applicant: Macquarie University, North Ryde (AU)

(72) Inventors: Lars Ittner, North Ryde (AU); Yazi Diana Ke, North Ryde (AU)

(21) Appl. No.: 17/754,135

(22) PCT Filed: Sep. 25, 2020
(86) PCT No.: PCT/AU2020/051022

§ 371 Date: Mar. 24, 2022

(30) Foreign Application Priority Data
Sep. 25, 2019 (AU) 2019 903587

(51) Int. Cl. A61K 31/519; A61P 25/28

(52) U.S. Cl. CPC A61K 31/519; A61P 25/28

(57) ABSTRACT

Provided herein are methods for treating, delaying the onset of, or ameliorating at least one symptom of, dementia associated with #-amyloid (A#) accumulation, and for improving memory in subjects suffering from dementia associated with A# accumulation, comprising administering to subjects in need thereof an effective amount of an inhibitor of LIMK1, wherein the inhibitor comprises a compound of Formula (I) as defined herein, or a pharmaceutically acceptable salt thereof. Also provided are methods for reducing A# toxicity in neurons, comprising exposing neurons to an effective amount of an inhibitor of LIMK1, wherein the inhibitor comprises a compound of Formula (I) as defined herein, or a pharmaceutically acceptable salt thereof.

### FIELD OF THE ART

The present disclosure relates generally to methods for the treatment of dementia associated with β-amyloid accumulation, in particular Alzheimer's disease, using inhibitors of LIM-domain kinase 1 (LIMK1) as defined herein.

### BACKGROUND

Alzheimer's disease is the most common form of senile and pre-senile dementia worldwide, accounting for more than 50% of all cases of dementia among people over 65 years of age. Alzheimer's disease is characterized clinically by the gradual and progressive decline in cognitive function, typically presenting as increasing loss of memory, intellectual capacity and disturbances in speech (dysphasia). On average, death occurs about nine years after diagnosis. The incidence of Alzheimer's disease increases dramatically with age, with estimates indicating that more than 50% of people worldwide over the age of 85 suffer from Alzheimer's disease.

Alzheimer's disease is characterized pathologically by deposition of β-amyloid peptide (Aβ) in extracellular plaques and accumulation of the microtubule binding protein tau in intra-neuronal neurofibrillary tangles. At the cellular level, neuronal connections are progressively compromised resulting in synaptic dysfunction and loss prior to loss of cells. These neuropathological changes are paralleled by the progressive loss of memory.

Aβ is a 38-43 amino acid peptide produced by cleavage from the neuronal transmembrane protein amyloid precursor protein (APP) by β- and γ-secretases. Aβ is released into the extracellular space where it aggregates to form plaques. AP is toxic to neurons, causing pore formation resulting in disruption of cellular calcium balance and loss of membrane potential, promoting apoptosis, causing synaptic loss, and disrupting the cytoskeleton. Increasing evidence suggests that it is soluble oligomers of Aβ that cause most Aβ toxicity and are responsible for cognitive dysfunction and decline.

Prognosis for sufferers of Alzheimer's disease is poor and treatments are limited. There is a clear need for the development of new methods for treating this debilitating disease and related forms of dementia and cognitive decline.

### SUMMARY OF THE DISCLOSURE

The present disclosure is predicated on the inventors' finding that genetic depletion and pharmacological inhibition of LIM-domain kinase 1 (LIMK1) improve memory deficits and neuronal network aberrations in a mouse model of Alzheimer's disease, and protects the mice from β-amyloid (Aβ) toxicity and excitotoxicity.

According to a first aspect of the present disclosure there is provided a method for treating, delaying the onset of, or ameliorating at least one symptom of, dementia associated with β-amyloid (Aβ) accumulation, the method comprising administering to a subject in need thereof an effective amount of an inhibitor of LIMK1, wherein the inhibitor comprises a compound of Formula (I) or a pharmaceutically acceptable salt thereof:

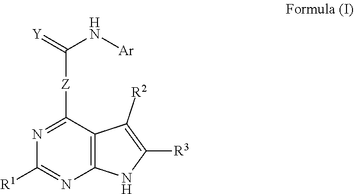

wherein:

Z is selected from the group consisting of optionally substituted cycloalkylene, optionally substituted arylene and optionally substituted aniline;

R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;

Y is selected from the group consisting of O, S, NCN, NCS and NSO₂Me;

Ar is selected from the group consisting of optionally substituted aryl, optionally substituted heteroaryl and optionally substituted heterocyclyl.

In some embodiments, Z is selected from one of the following structures:

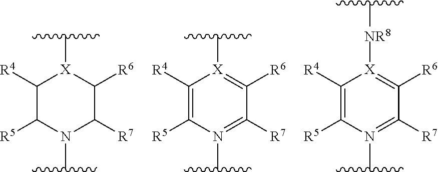

wherein:

R⁴, R⁵, R⁶ and R⁷ have the same definition as R² and R³ above;

X is CH or N; and

R⁸ is H or optionally substituted alkyl.

In a particular embodiment, the compound of Formula (I) is the compound of Formula (Ia) or a pharmaceutically acceptable salt thereof:

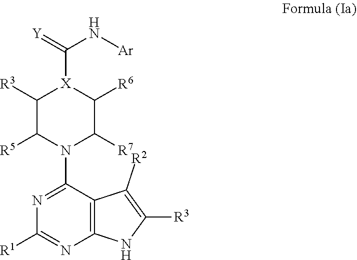

wherein variables R¹ to R⁷, X, Y and Ar are as defined above.

In an embodiment, the compound of Formula (I) is the compound of Formula (Ia), or a pharmaceutically acceptable salt thereof, wherein:

R¹, R³, R⁵, R⁶ and R⁷ are H;

R² is methyl;

R³ is (S)-methyl;

X is N;

Y is NCN; and

Ar is 3-bromophenyl.

The compound may be selective inhibitor of LIMK1. The compound may be a specific inhibitor of LIMK1.

In an embodiment, the compound of Formula (Ia) has the following structure:

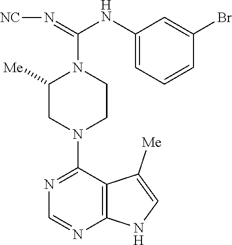 [SMILES: Cc1c[nH]c2ncnc(N3CCN(/C(=N\C#N)Nc4cccc(Br)c4)[C@@H](C)C3)c12]

In a particular embodiment, the dementia is Alzheimer's disease.

The at least one symptom of the dementia may comprise a clinical or pathological symptom. In particular embodiments, the at least one symptom may comprise memory deficits and/or aberrations or disintegration of neuronal networks. Aberrations or disintegration of neuronal networks may be associated with excitotoxicity or Aβ toxicity.

According to a second aspect of the present disclosure there is provided a method for improving memory in a subject suffering from dementia associated with β-amyloid (Aβ) accumulation, the method comprising administering to a subject in need thereof an effective amount of an inhibitor of LIMK1, wherein the inhibitor comprises a compound of Formula (I) as defined above or a pharmaceutically acceptable salt thereof.

According to a third aspect of the present disclosure there is provided a method for reducing Aβ toxicity in neurons, the method comprising exposing neurons to an effective amount of an inhibitor of LIMK1, wherein the inhibitor comprises a compound of Formula (I) as defined above or a pharmaceutically acceptable salt thereof.

A fourth aspect of the disclosure provides the use of an inhibitor of LIMK1, wherein the inhibitor comprises a compound of Formula (I) as defined above or a pharmaceutically acceptable salt thereof in the manufacture of a medicament for treating, delaying the onset of, or ameliorating at least one symptom of, dementia associated with β-amyloid (Aβ) accumulation.

A fifth aspect of the disclosure provides the use of an inhibitor of LIMK1, wherein the inhibitor comprises a compound of Formula (I) as defined above or a pharmaceutically acceptable salt thereof in the manufacture of a medicament for improving memory in a subject suffering from dementia associated with β-amyloid (Aβ) accumulation.

A sixth aspect of the disclosure provides the use of an inhibitor of LIMK1, wherein the inhibitor comprises a compound of Formula (I) as defined above or a pharmaceutically acceptable salt thereof in the manufacture of a medicament for reducing Aβ toxicity in neurons.

### BRIEF DESCRIPTION OF THE DRAWINGS

Aspects and embodiments of the present disclosure are described herein, by way of non-limiting example only, with reference to the following drawings.

FIG. 1. Limk1 depletion prevented memory deficits and neuronal network aberrations in a mouse model of Alzheimer's disease. (A) Left graph: Temporal improvement of escape latency was delayed in Limk1^{-/-} mice and severely compromised in APP transgenic APP23 (APP23/Limk^{1+/+}) mice as compared to non-mutant littermates (Limk1^{+/+}). APP23/Limk1^{-/-} mice preformed similar to Limk1^{-/-} mice indicative of improved learning. Right graph: Significant memory consolidation deficits during probe trials in Limk1^{-/-}, App23/Limk1^{+/+} and APP23/Limk1^{-/-} mice as compared with Limk1^{+/+} controls. n=8-10, *p<0.05, **p<0.01 (Student's t tests). From left to right: Limk1^{+/+}; Limk1^{-/-}; APP23/Limk^{1+/+}; APP23/Limk1^{-/-}. (B) APP23/Limk1+/+ mice fail to learn the location of the escape platform 24 hours after acquisition in the water T maze, while APP23/Limk1^{-/-} mice perform like Limk1^{+/+} and Limk1^{+/+} with significantly less errors after 24 hours. n=7-8, *p<0.05, **p<0.01 (ANOVA). For each of Limk1^{+/+}, Limk1^{-/-}, APP23/Limk^{1+/+} and APP23/Limk1^{-/-}: left hand column is at acquisition; middle column is after 2 hours post acquisition; and right hand column is after 24 hours. (C) Left graph: Significantly increased number of spikes per minute in APP transgenic APP23 mice (APP23/Limk1^{+/+}) as compared with non-transgenic (Limk1^{+/+}) and Limk1 knockout (Limk1^{-/-}) littermates during telemetric hippocampal electroencephalography (EEG) recordings in freely moving mice. Spike frequency was significantly lower in EEG recordings of APP23/Limk1^{-/-} mice as compared with APP23/Limk1^{+/+} littermates, and not significantly (ns) different from Limk1^{+/+} and Limk1^{-/-} controls. n=11, *p<0.05, ***p<0.001 (Student's t tests). Right graph: Display of spike frequency in EEG recordings of individual Limk1^{+/+}, Limk1^{-/-}, APP23/Limk1^{+/+} and APP23/Limk1^{-/-} mice statistically analysed in the left graph. (D) Significantly reduced modulation index of cross frequency coupling in APP23/Limk1^{+/+} mice as compared with APP23/Limk1⁻⁻ littermates and Limk1^{+/+} and Limk1^{-/-} controls. n=8, ***p<0.001 (Student's t tests). For (C) and (D), from left to right: Limk1^{+/+}; Limk1^{-/-}; APP23/Limk1+/+; APP23/Limk1^{-/-}. (E) Significantly disrupted amplitude phase in EEG recordings of APP23/Limk1^{+/+} mice as compared with APP23/Limk1^{-/-} littermates and Limk1^{+/+} and Limk1^{-/-} controls. n=8, *p<0.05, **p<0.01 (ANOVA).

FIG. 2. Increased Limk 1 activity in human Alzheimer's disease and a mouse model of Alzheimer's disease. (A) Representative immunohistochemical staining of human Alzheimer's disease (left) and human mutant APP transgenic APP23 mouse (right) brains. Cells with accumulation of the phosphorylated LIMK1 substrate ADF/cofilin (p-ADF/cofilin; red; arrows) around amyloid-β (Aβ) plaques (green) stained with the Aβ antibody 6E10. Nuclei were stained with DAPI. Scale bars, 50 μm. (B) Quantification of markedly reduced numbers of ADF/cofilin aggregates in the hippocampus of aged APP23/Limk1^{-/-} mice compared with APP23 littermates.

FIG. 3. LIMK1 inhibitor (LIMKi) efficiently inhibits LIMK1 activity. In vitro LIMK1 activity assay demonstrates inhibition of the kinase by LIMKi at all concentrations tested (100 μm, 10 μm, 1 μm, 0.1 μm). DMSO (solvent) did not inhibit LIMK1 activity.

FIG. 4. LIMK1 inhibition reduced susceptibility to induced seizures and mitigated neuronal network hypersynchronicity and memory deficits in a mouse model of Alzheimer's disease. (A) Excitotoxic seizures were induced by acute intraperitoneal injection of 50 mg/kg body weight pentylenetetrazole (PTZ) followed by timing and scoring (0=no seizures to 7=terminal status epilepticus) of seizure development. Mice were treated with vehicle or 10 mg/kg body weight LIMK1 inhibitor (LIMKi) intraperitoneally 30 minutes prior to seizure induction. Left graph: Delayed latency to develop lower grade seizures (i.e. score<5) in LIMKi-treated mice (darker line) compared with vehicle controls. Only vehicle-treated mice develop severe seizure stages over time (i.e. score=5). Right graph: Significantly reduced seizure severity in LIMKi-treated mice (right hand column) as compared with vehicle controls. n=9, **p<0.001 (Student's t test). (B) Compound hippocampal electroencephalography (EEG) recordings over 4 hours in non-transgenic (wt) and APP transgenic (APP23) mice including 2 hours before (Pre) and 2 hours after (Post) LIMKi or vehicle administration. Time of intraperitoneal LIMKi and vehicle administration is indicated with an arrow. Left: No overt spike activity pre and post vehicle or LIMKi injection in wt mice. Frequent spikes pre and post vehicle administration in APP23 mice. In contrast, LIMKi injection mitigates spike activity in APP23 post injection. Right graphs: No significant changes in spike frequency in vehicle-treated APP23 and significantly reduced spike frequency in LIMKi treated APP23 mice when comparing pre and post spike numbers. n=8, *p<0.05 (Student's t test). (C) Significantly reduced modulation index of cross frequency coupling of APP23 mice (vehicle) was corrected 1 hours after LIMKi administration and no longer different from wt controls (vehicle/LIMKi-treated) n=8, **p<0.01 (Student's t tests). (D) Significantly disrupted amplitude phase in EEG recordings of APP23 mice (vehicle) was corrected 1 hour after LIMKi delivery and no longer different from wt controls (vehicle/LIMKi-treated). n=8, *p<0.05 (ANOVA). (E) Chronic Limkl inhibition reverts memory deficits in AD mice. APP23 mice develop memory deficits in the Morris water maze (MWM) paradigm at 3 months of age. Treatment with 10 mg/kg LIMKi i.p. (3-4 times per week) was initiated at 3 months of age and continued for =4 months before MWM testing at 7 months of age. Left graph: Vehicle-treated APP23 mice showed delayed learning as compared with vehicle-treated controls. LIMKi-treated controls showed moderately delayed learning, however, LIMKi treatment improved learning of APP23 mice to vehicle control levels. Right Graph: Vehicle-treated APP23 spent significantly less time in the target quadrant during MWM probe trials, indicative of defective memory formation. LIMKi-treated APP23 showed no significant differences to controls. n=8-10, *p<0.05 (ANOVA). For bar graphs in (C) and (E), from left to right: non-Tg +vehicle; non-Tg+LIMKi; APP23+vehicle; APP23+LIMKi.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the evet that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

The term “inhibitor” as used herein refers to an agent that decreases or inhibits at least one function or biological activity of a target molecule, e.g. LIMK1, either directly or indirectly. The term “selective” and grammatical variants thereof are used herein to refer to agents that inhibit a target molecule without substantially inhibiting the function of another molecule.

In the context of the present disclosure, the terms “inhibiting” and grammatical equivalents do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably. Similarly, the terms “inhibit”, “decrease” and “reduce” may be used interchangeably, in reference to the level of, or a value for, a substance, phenomenon, function or activity in a second sample or at a second timepoint that is lower than the level of, or value for, the substance, phenomenon, function or activity in a first sample or at a first timepoint. The reduction may be determined or measured subjectively or objectively, and may be subject to an art-accepted statistical method of analysis.

Use of the term “associated with” herein describes a temporal, physical or spatial relationship between events, symptoms or pathologies. Thus for example, in the context of the present disclosure dementia associated with Aβ accumulation means that the dementia is at least partially characterized by, or results from, either directly or indirectly, the accumulation, typically extracellular accumulation of Aβ. The dementia may occur or begin at the time of accumulation of the Aβ. Alternatively, the Aβ accumulation and the dementia may be temporally spaced such that the onset of the dementia is minutes, hours, days, weeks, months or years after the accumulation of Aβ begins.

As used herein the terms “treating”, “treatment”, and grammatical equivalents refer to any and all uses which remedy the stated neurodegenerative disease, prevent, retard or delay the establishment of the disease, or otherwise prevent, hinder, retard, or reverse the progression of the disease. Thus the terms “treating” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein the term “effective amount” includes within its meaning a non-toxic but sufficient amount or dose of an agent or compound to provide the desired effect. The exact amount or dose required will vary from subject to subject depending on factors such as the species being treated, the age, size, weight and general condition of the subject, the severity of the disease or condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The LIM-domain family of protein kinases includes LIM-domain kinase 1 (LIMK1) and LIM-domain kinase 2 (LIMK2). The LIM kinases are serine/threonine protein kinases which bind actin, influencing the architecture of the actin cytoskeleton by regulating the activity of cofilin proteins through phosphorylation. Most highly expressed in the brain, neuronal LIMK1 is an established regulator of synaptic morphology. Prior to the present invention little was known about the functional role of LIMK1 in neurodegenerative disease. Human LIMK1 is a 647 amino acid protein (UniProt protein database Accession No. P53667) produced from the LIMK1 gene encoded on chromosome 7. Alternative splicing produces four isoforms of LIMK1.

As exemplified herein, the inventors have demonstrated the effects of Limk1 depletion and LIMK1 inhibition on memory performance, neuronal network activity and neuropathological changes in Afl formation, in transgenic mice. The data described herein establish a new role of LIMK1 in Alzheimer's disease and provide a novel potential therapeutic approach to improve functional deficits in Alzheimer's disease and related forms of dementia by inhibition of LIMK1.

In one aspect the present disclosure provides a method for treating, delaying the onset of, or ameliorating at least one symptom of, dementia associated with β-amyloid (Aβ) accumulation, the method comprising administering to a subject in need thereof an effective amount of an inhibitor of LIMK1, wherein the inhibitor comprises a compound of Formula (I) or a pharmaceutically acceptable salt thereof:

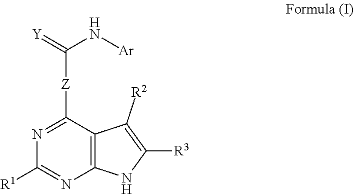

wherein:

Z is selected from the group consisting of optionally substituted cycloalkylene, optionally substituted arylene and optionally substituted aniline;

R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;

Y is selected from the group consisting of O, S, NCN, NCS and NSO₂Me;

Ar is selected from the group consisting of optionally substituted aryl, optionally substituted heteroaryl and optionally substituted heterocyclyl.

In some embodiments, Z is selected from one of the following structures:

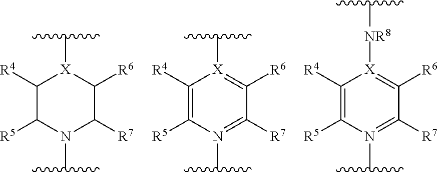

wherein:

R⁴, R⁵, R⁶ and R⁷ have the same definition as R² and R³ above;

X is CH or N; and

R⁸ is H or optionally substituted alkyl.

“Alkyl” refers to a monovalent alkyl groups that may be straight chained or branched, and preferably have from 1 to 10 carbon atoms, or more preferably 1 to 6 carbon atoms. Examples of such groups include methyl, ethyl, n-isopropyl, iso-propyl, n-butyl, iso-butyl, n-hexyl, and the like.

“Alkenyl” refers to a monovalent aliphatic carbocyclic group having at least one carbon-carbon double bond and which may be straight chained or branched, preferably having from 2 to 10 carbon atoms. Examples of such groups include a vinyl or ethenyl group (—CH═CH₂), n-propenyl (—CH₂CH═CH₂), iso-propenyl (—C(CH₃)═CH₂), but-2-enyl (—CH₂CH═CHCH₃), and the like.

“Alkynyl” refers to a monovalent aliphatic carbocyclic group having at least one carbon-carbon triple bond and which may be straight chained or branched, preferably having from 2 to 10 carbon atoms. Examples of such groups include an acetylene or ethynyl group (—C≡CH), propargyl (—CH₂C≡CH), and the like.

“Aryl” refers to a monovalent unsaturated aromatic carbocyclic group having a single ring (e.g. phenyl) or multiple condensed rings (e.g. naphthyl, anthracenyl), preferably having from 6 to 14 carbon atoms. Examples of aryl groups include phenyl, naphthyl, anthracenyl and the like.

“Alkoxy” and “aryloxy” refers to the groups “—O-alkyl” and “—O-aryl”, respectively, wherein the alkyl and aryl groups are described above.

“Halogen” refers to the groups fluoro, chloro, bromo and iodo.

“Heteroaryl” refers to a monovalent aromatic carbocyclic group, preferably having from 6 to 14 carbon atoms and 1 to 4 heteroatoms, wherein the heteroatoms are within the ring and are selected independently from oxygen, nitrogen and sulfur. Such heteroaryl groups can have a single ring (e.g. pyridyl, pyrrolyl or furyl) or multiple condensed rings (e.g. indolyl and benzofuryl).

“Heterocyclyl” refers to a monovalent saturated or unsaturated group having a single ring or multiple condensed rings, preferably having from 4 to 10 carbon atoms and from 1 to 4 heteroatoms, wherein the heteroatoms are selected independently from nitrogen, sulfur, oxygen, selenium and phosphorus.

Examples of heterocyclyl and heteroaryl groups include, but are not limited to pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo [b]thiophene, thiazole, thiazolidine, thiophene, benzo[b]thiophene, morpholino, piperidinyl, pyrrolidine, tetrahydrofuranyl, and the like.

As used herein, the term “optionally substituted” in relation to a particular group is taken to mean that the group may or may not be further substituted with one or more groups selected from hydroxyl, acyl, alkyl, alkoxy, alkenyl, alkenyloxy, alkynyl, alkynyloxy, amino, aminoacyl, alkylaryl, aryl, aryloxy, carboxyl, acylamino, cyano, halogen, nitro, sulphate, phosphate, phosphine, heteroaryl, heterocyclyl, oxyacyl, oxyacylamino, aminoacyloxy, trihalomethyl, and the like.

Examples of particularly suitable optional substituents include F, Cl, Br, I, CH₃, CH₂CH₃, OH, OCH₃, CF₃, OCF₃, NO₂, NH₂, COCH₃ and CN.

As used herein, the term “pharmaceutically acceptable salts” refers to salts that retain the desired biological activity of the parent compound, and include pharmaceutically acceptable acid addition salts and base addition salts. Suitable pharmaceutically acceptable acid addition salts of compounds of Formula (I) may be prepared from an inorganic acid or an organic acid. Examples of an inorganic acid include hydrochloric acid, sulphuric acid and phosphoric acid. Examples of organic acids include aliphatic, cycloaliphatic, aromatic, heterocyclic carboxylic and sulfonic organic acids, such as, formic, acetic, proprionic, succinic, glycolic, gluronic, lactic, malic, tartaric, citric, fumaric, maleic, alkylsulfonic and arylsulfonic acids. Where the compound of Formula (I) is a solid, the compounds and salts thereof may exist in one or more different crystalline or polymorphic forms, all of which are intended to be within the scope of Formula (I).

In a particular embodiment, the compound of Formula (I) is the compound of Formula (Ia):

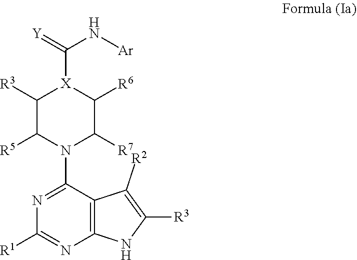

or a pharmaceutically acceptable salt thereof, wherein variables R¹ to R⁷, X, Y and Ar are as defined above.

In an embodiment, the compound of Formula (I) is the compound of Formula (Ia), or a pharmaceutically acceptable salt thereof, wherein:

R¹, R³, R⁵, R⁶ and R⁷ are H;

R² is methyl;

R³ is (S)-methyl;

X is N;

Y is NCN; and

Ar is 3-bromophenyl.

In an exemplary embodiment of the present disclosure, the compound of Formula (Ia) has the following structure (also referred to herein as LIMKi):

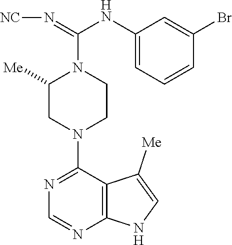 [SMILES: Cc1c[nH]c2ncnc(N3CCN(/C(=N\C#N)Nc4cccc(Br)c4)[C@@H](C)C3)c12]

The inhibitor may be a specific inhibitor of LIMK1 or may be selective for LIMK1. Thus, the inhibitor may also display inhibitory activity against LIMK2. The inhibitor may display inhibitory activity against one or more isoforms of LIMK1.

Embodiments of the present disclosure are applicable to the treatment of dementia associated with β-amyloid (Aβ) accumulation. The treatment may result in improvements in one or more clinical manifestations of the dementia, such as improvements in memory, including a reduction or prevention in memory deficits or memory impairment associated with the dementia. The treatment may result in improvements in relation to one or more pathological characteristics of dementia, for example, a reduction in Aβ toxicity or in aberrations or disintegration of neuronal networks.

Typically the dementia is Alzheimer's disease. Alternatively, the dementia may comprise or be associated with amyloidopathy (Alzheimer's dementia) or cerebral amyloid angiopathy. The subject may be diagnosed with the dementia, or may experience one or more symptoms of the dementia. A subject with dementia may be asymptomatic such that a treatment of the present disclosure delays the onset of the disease or a symptom thereof. Symptoms of the dementia include memory deficits and neuronal aberrations or disintegrations characteristic of or associated with the disease. The subject may be susceptible to or at risk of developing the dementia.

Embodiments of the present disclosure contemplates the delivery of LIMK1 inhibitors to subjects in need of treatment by any suitable means, and typically in the form of pharmaceutical compositions, which compositions may comprise one or more pharmaceutically acceptable carriers, excipients or diluents. Such compositions may be administered in any convenient or suitable route such as by parenteral (e.g. intraperitoneal, subcutaneous, intraarterial, intravenous, intramuscular, intrathecal, intracerebral, intraocular), oral (including sublingual), nasal, transmucosal or topical routes. In circumstances where it is required that appropriate concentrations of the molecules are delivered directly to the site in the body to be treated, administration may be regional rather than systemic. Regional administration provides the capability of delivering very high local concentrations of the molecules to the required site and thus is suitable for achieving the desired therapeutic or preventative effect whilst avoiding exposure of other organs of the body to the vectors and molecules and thereby potentially reducing side effects.

As will be appreciated by those skilled in the art, the choice of pharmaceutically acceptable carrier or diluent will be dependent on the route of administration and on the nature of the condition and subject to be treated. The particular carrier or diluent and route of administration may be readily determined by a person skilled in the art. The carrier or diluent and route of administration should be carefully selected to ensure that the activity of the compound is not depleted during preparation of the formulation and the compound is able to reach the site of action intact.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and me thylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

A person skilled in the art will readily be able to determine appropriate formulations for the compound to be administered using conventional approaches. Techniques for formulation and administration may be found in, for example, Remington (1980) Remington's Pharmaceutical Sciences, Mack Publishing Co., Easton, Pa., latest edition; and Niazi (2009) Handbook of Pharmaceutical Manufacturing Formulations, Informa Healthcare, New York, second edition, the entire contents of which are incorporated by reference.

Identification of preferred pH ranges (where appropriate) and suitable excipients is routine in the art, for example, as described in Katdare and Chaubel (2006) Excipient Development for Pharmaceutical, Biotechnology and Drug Delivery Systems (CRC Press).

In some embodiments, the compound of Formula I is formulated for oral administration in a dosage form such as a tablet, pill, capsule, liquid, gel, syrup, slurry, suspension, lozenge and the like for oral ingestion by a subject. In particular embodiments, the compound of Formula I is formulated for oral administration in a solid dosage form, such as a tablet, pill, lozenge or capsule. In such embodiments, the pharmaceutically acceptable carrier may comprise a number of excipients including, but not limited to, a diluent, disintegrant, binder, lubricant, and the like.

Suitable diluents (also referred to as “fillers”) include, but are not limited to, lactose (including lactose monohydrate, spray-dried monohydrate, anhydrous, etc.), mannitol, xylitol, dextrose, sucrose, sorbitol, compressible sugar, isomalt, microcrystalline cellulose, powdered cellulose, starch, pregelatinised starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose, silicates (e.g. silicon dioxide), polyvinyl alcohol, talc, and combinations thereof.

Suitable disintegrants include, but are not limited to, sodium carboxymethyl cellulose, pregelatinised starch, calcium carboxymethyl cellulose, croscarmellose sodium, crospovidone, polyvinylpyrrolidone, methylcellulose, sodium starch glycolate, microcrystalline cellulose, lower alkyl-substituted hydroxypropyl cellulose, starch, sodium alginate and combinations thereof. Suitable binders include, but are not limited to, microcrystalline cellulose, gelatine, sugars, polyethylene glycol, natural and synthetic gums, polyvinylpyrrolidone, pregelatinized starch, hydroxypropyl cellulose, hydroxypropyl methylcellulose and combinations thereof. Suitable lubricants include, but are not limited to, magnesium stearate, calcium stearate, zinc stearate, sodium stearyl fumarate, polyethylene glycol and combinations thereof.

Pharmaceutical formulations for parenteral administration include aqueous solutions of the compound of Formula I in water-soluble form. Additionally, suspensions of the compound of Formula I may be prepared as appropriate oily injection suspensions. Suitable lipophilic solvents or carriers include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilisers or agents that increase the solubility of the compounds to allow for the preparation of highly concentrated solutions.

Sterile solutions may be prepared by combining the compound in the required amount in the appropriate solvent with other excipients as described above as required, followed by sterilization, such as filtration. Generally, dispersions are prepared by incorporating the various sterilised active compounds into a sterile vehicle which contains the basic dispersion medium and the required excipients as described above. Sterile dry powders may be prepared by vacuum- or freeze-drying a sterile solution comprising the active compounds and other required excipients as described above.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination. For injection, compositions may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

It will be understood that the specific dose level of a composition of the invention for any particular subject will depend upon a variety of factors including, for example, the activity of the inhibitor employed, the half-life of the inhibitor, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

In exemplary embodiments of the present disclosure it is contemplated that the inhibitor may be administered to a subject daily or less than daily, for example every second day or every third day for the duration of treatment required to achieve the desired outcome. Administration may be continuous, for example on a daily basis or every second day, or may be intermittent with spacing between administrations determined by the treating medical professional depending on response of the subject to treatment and progress of the subject during the course of treatment.

The present invention contemplates combination therapies, wherein LIMK1 inhibitors as described herein are coadministered with other suitable agents that may facilitate the desired therapeutic or prophylactic outcome. By “coadministered” is meant simultaneous administration in the same formulation or in two different formulations via the same or different routes or sequential administration by the same or different routes. By “simultaneously” is meant that the active agents are administered at substantially the same time. By “sequential” administration is meant a time difference of from seconds, minutes, hours or days between the administration of the agents. Administration may be in any order.

Each embodiment described herein is to be applied mutatis mutandis to each and every embodiment unless specifically stated otherwise.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

### General Methods

Mice. APP23 express human Swedish mutant (K595N/M596L) amyloid-13 precursor protein (APP) in neurons (Sturchler-Pierrat et al., 1997, Proc Natl Acad Sci USA 94, 13287-13292). Limk1^{+/+} mice have been reported previously (Meng et al., 2002, Neuron 35, 121-133). All mice were on a pure C57B1/6 background. Mice were housed in standard individually ventilated cages on a 12 hour light/dark cycle with access to standard chow and water ad libitum. Mice of both genders were used unless otherwise indicated. Investigators were blinded to genotypes until after completion of data analysis. All experiments were approved by the Animal Ethics Committee of Macquarie University.

Memory testing. Memory of mice was assessed in the water T maze paradigm and Morris water maze following published protocols (Ittner et al., 2010, Cell 142, 387-397; Ke et al., 2015, Acta Neuropathol 130, 661-678). Chronic treatment of mice was done by 3-4 times per week intraperitoneal injection of 10 mg/kg LIMKi or vehicle (0.5% hydroxypropyl methylcellulose 606 (HPMC 606), 0.5% polyvinylpyrrolidone k17 (PVP k17) and 0.1% tween 80) starting at 3 months of age and continued until 7 months of age. All mice were acclimatized to the test rooms prior to testing.

Electroencephalography. Hippocampal EEG recordings were done with implanted telemetric electrodes (D.S.I.) as previously described (Ittner et al., 2014, Acta neuropathologica communications 2, 149). All recordings were done with mice individually housed in their home cages one week after implantation of transmitters. Compound administration was done intraperitoneally during transient restraining of mice. Vehicle for the LIMK1 inhibitor LIMKi contained hydroxypropyl methylcellulose 606 (HPMC 606) with polyvinylpyrrolidone k17 (PVP k17) and tween 80 in a proportion of 0.5%, 0.5%, 0.1%, respectively. Spike analysis was done with the Neuroscore software module (D.S.I.) as previously described (Ittner et al., 2016, Science 354, 904-908). Cross frequency coupling of theta and gamma waves was performed as previously described (Ittner et al., 2016, Science 354, 904-908).

Tissue staining. Staining of paraffin embedded brain tissue was performed as previously described (Ittner et al., 2010, Cell 142, 387-397). Primary antibodies were to Aβ (6E10; Covance), p-ADF/cofilin (Sigma) and ADF/cofilin (Sigma). Nuclei were visualized with DAPI (Molecular Probes). ADF aggregates were counted on serial hippocampal sections.

LIMK1 activity assay. LIMK1 activity in the absence or presence of different concentrations of LIMKi (or DMSO) was determine with a commercial assay following the manufacturer's instructions (Promega).

Seizure model. Excitotoxic seizure were induced in mice by intraperitoneal injection of 50 mg/kg body weight pentylenetetrazole followed by observation in a square area (40×40 cm). Scoring of seizures was done as previously described (Ittner et al., 2010, Cell 142, 387-397). For LIMKi-treated mice, scoring was adjusted by combining the severe seizure scores 5 to 7 into a single score of 5. For both scales, minor to moderate seizures are reflected by scores <5.

Statistical analysis. All statistical analyses were done with the Prism 7 software (GraphPad). All values are presented as mean and standard error of the mean. P values<0.05 were considered significant.

### EXAMPLE 1

### Limk1 Depletion Prevents Memory Deficits and Neuronal Network Aberrations

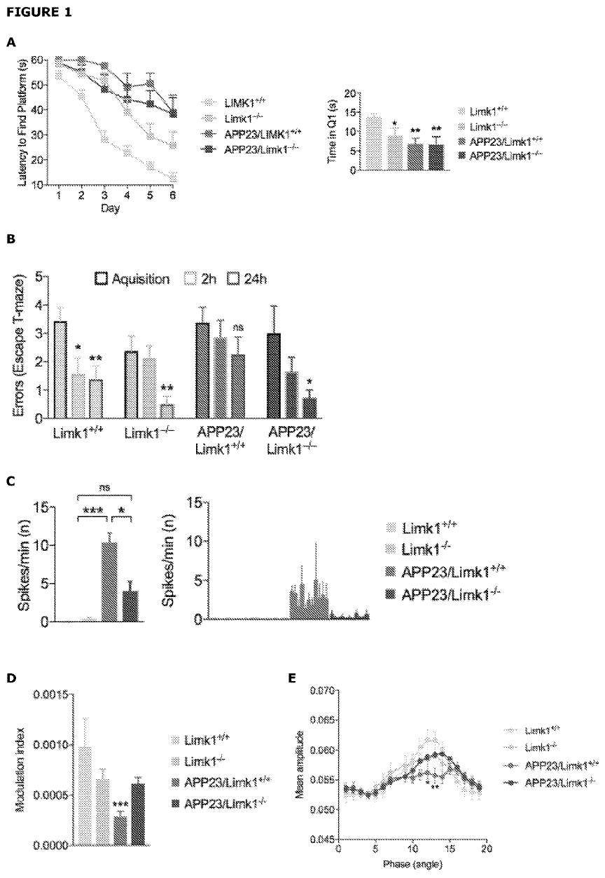

Alzheimer's disease mouse models with transgenic expression of human mutant APP present with memory deficits, AP pathology and premature mortality, which has been associated with excitotoxicity (Ittner and Gotz, 2011, Nat Rev Neurosci 12:67-72). Limk1⁻¹⁻ mice have increased long-term potentiation, but no fundamental changes to memory formation (Meng et al., 2002, Neuron 35:121-133). To determine whether Limk1 contributes functionally to the deficits in Alzheimer's disease mice, the inventors crossed Aβ-forming APP23 mice with a Limk1⁻⁻ strain to obtain APP23/Limk1^{-/-} mice. Memory was assessed by standard Morris water maze (MWM) and T-maze testing. APP23 presented with profound learning deficits in the MWM paradigm as compared to non-transgenic littermate controls, while Limk1^{-/-} mice showed a trend towards delayed learning. Interestingly, APP23/Limk1^{-/-} mice showed improved learning profiles comparable to those of Limkl mice (FIG. 1A). These findings were consolidated by T-maze testing. Performance during acquisition was comparable for all genotypes, but APP23 failed to memorize the target arm in subsequent testing sessions, while APP23/Limk1^{-/-}, Link1⁻⁺ mice and non-transgenic mice presented comparable memory in this simple task (FIG. 1B). Taken together, Limk1-depletion protected against severe memory deficits of APP23 mice.

APP23 mice present with non-convulsive, silent seizure activity during electroencephalography (EEG) recordings, as well as disrupted cross frequency coupling (CFC) of θ phase modulation of γ power during no-spike episodes of EEG recordings (Ittner et al., 2014, Acta Neuropath Comm 2:149), a modality linked to memory formation including in humans. The inventors implanted APP23/Limk1^{-/-} mice, as well as APP23/Limk1^{+/+}, Limk1^{-/-} and Limk1^{+/+} littermates with telemetric EEG transmitters off hippocampal electrodes for recording in freely moving mice. APP23/Limk1^{+/+} mice presented with frequent hypersynchronous discharges during EEG recordings, while there were virtually no such events detected in Limk1^{-/-} and Limk1^{+/+} littermate recordings (FIG. 1C). For comparison, APP23/Limk1^{-/-} mice showed significantly reduced numbers of spikes that were not significantly different from controls. CFC during spike-free episodes was disrupted in APP23/Limk1^{+/+} mice (data not shown). In contrast, APP23/Limk1^{-/-} mice showed the same CFC at 8 Hz as detected in Limk1^{-/-} and Limk1^{+/+} littermate recordings. Accordingly, the significantly reduced modulation index (FIG. 1D) and amplitude phase (FIG. 1E) of APP23/Limk1^{+/+} mice was normalized in APP23/Limk1^{-/-} mice to levels of Limk1^{-/-} and Limk1^{+/+} littermate recordings Taken together, neuronal network aberrations of APP23 mice were mitigated by knocking out Limk1.

### EXAMPLE 2

### Pathology and Reduced Aβ Toxicity in Limk1-deficient APP23 Mice

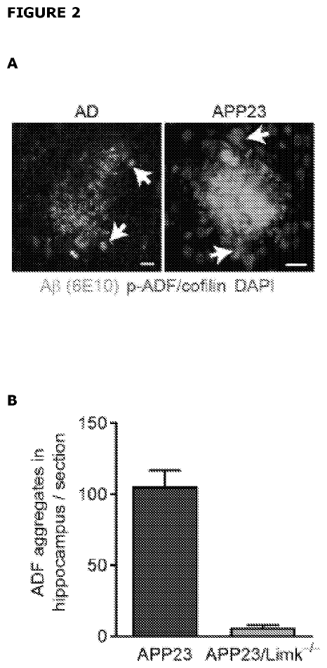

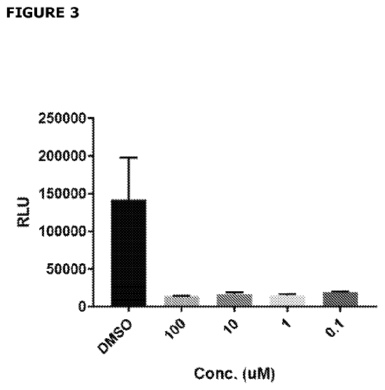

In brain sections from individuals with Alzheimer's disease, neurons in the proximity of Aβ plaques stain markedly for phosphorylated ADF/cofilin (p-ADF/cofilin), the downstream target of LIMK1 (FIG. 2A). Similarly, neurons in the proximity of Aβ plaques in the brains of APP23 mice labelled for p-ADF/cofilin. No similar intensive labelling of neurons was found in non-transgenic mouse and human controls (data not shown). Staining APP231Limk1^{+/+} brains with ADF/cofilin antibodies revealed frequent rod-like structures in the hippocampus (FIG. 2B). In contrast, virtually no such rod-like structures were found in APP231Limk1^{-/31} brains. Limk1^{-/-} and Limk1^{+/+} brains harbored no such inclusions (data not shown).

### EXAMPLE 3

### Pharmacological LIMK1 Inhibition Protects Neurons from Aβ Toxicity and Mitigates Network Aberrations in APP23 Mice

Given the improvements demonstrated in APP23/Limk1^{-/-} mice (Example 1), the inventors determined whether treatment with a LIMK1 inhibitor could similarly prevent Aβ toxicity and improve deficits of APP23 mice. C57B^{1/6} primary mouse neurons were treated with Compound 1 shown below (also referred to herein as LIMKi) together with 0.05 μM oligomeric Aβ (oAβ ) and determined loss of dendritic spines and cell viability.

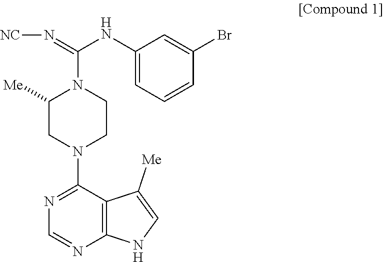 [SMILES: Cc1c[nH]c2ncnc(N3CCN(/C(=N\C#N)Nc4cccc(Br)c4)[C@@H](C)C3)c12]

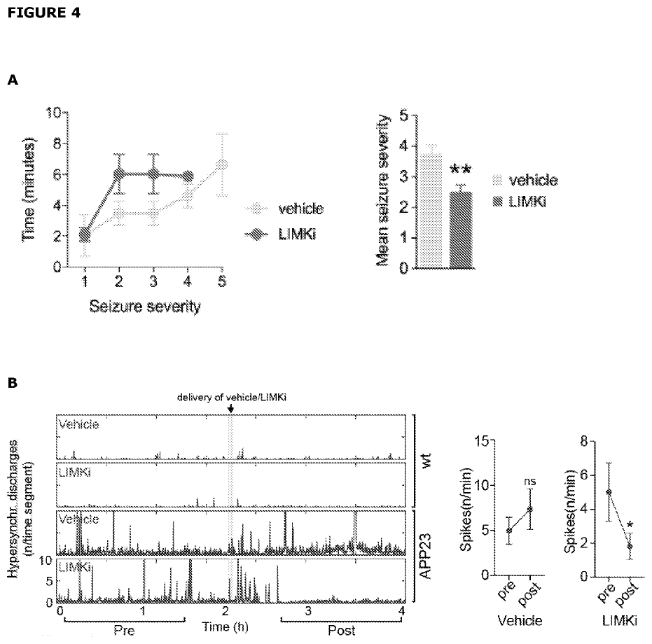

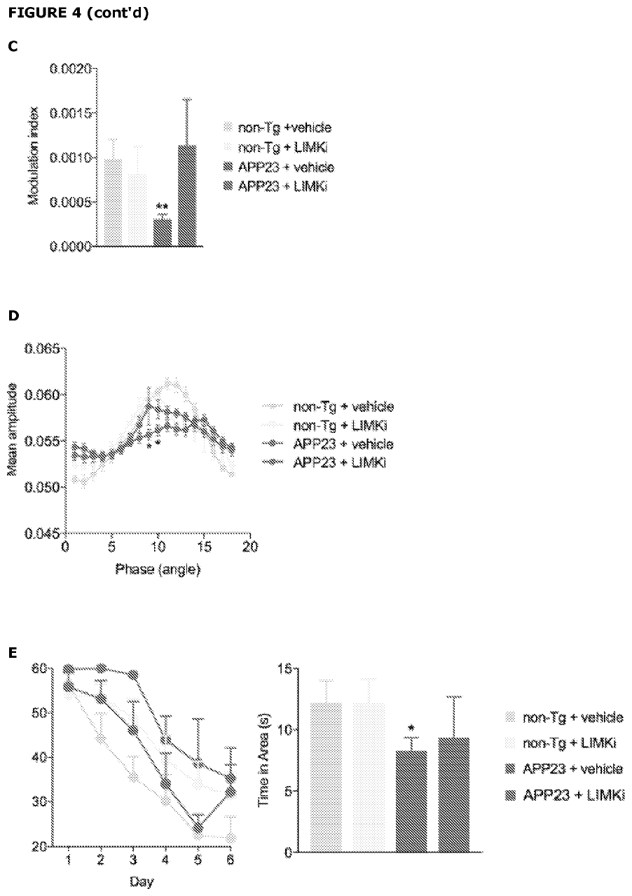

Aβ treated neurons presented with loss of spines and reduced viability. However, pre-treatment with Compound 1 conferred a dose-dependent protection from Aβ-induced dendritic spine loss and death of cultured neurons. Next, the inventors determined the potential short-term effects of Compound 1 in vivo. Administration of 10 mg/kg Compound 1 30 minutes prior to inducing excitotoxic seizures with PTZ, significantly reduced mean seizure severity and increased the latency to develop severe seizures in C57B^{1/6} mice (FIG. 4A). Next, the inventors tested the effects of acute Compound 1 administration on neuronal network activity in APP23 mice. Recordings prior to drug administration showed frequent hypersynchronous discharges in APP23 mice (FIG. 4B). Similarly, continued recordings over 2 hours after vehicle injections showed high spike activity. However, 30 minutes after Compound 1 administration, hypersynchronicity significantly decreased in Compound 1 treated APP23 mice compared to vehicle-treated APP23 mice and compared to recordings prior to injections (FIG. 4B). Furthermore, CFC 1 hour after Compound 1 administration was re-established (data not shown). The significantly reduced modulation index (FIG. 4C) and amplitude phase (FIG. 4D) was normalized by Compound 1 administration in APP23 mice. Thus, inhibition of LIMK1 with Compound 1 conferred protection from Aβ toxicity in neurons and from excitotoxicity in vivo. Furthermore, treatment of APP23 with Compound 1 corrected network aberrations including CFC.

### EXAMPLE 4

### Pharmacological LIMK1 Inhibition Improves Memory Deficits in APP23 Mice

Encouraged by the short-term effects of Compound 1 in neurons and APP23 mice (Example 3), the inventors tested whether long-term treatment could improve memory deficits in APP23 mice. 3-month old APP23 mice were treated for four months before being subjected to memory testing in the MWM. APP23 mice present with significant memory deficits at 3 months of age, that progress with age (Ittner et al., 2016, Science 354:904-908). An intermittent treatment schedule was chosen to avoid potential inhibition of astrocyte-mediated AP clearance that could mask therapeutic effects. At 7 months of age, vehicle-treated APP23 mice presented with significant memory deficits as compared to vehicle-treated non-transgenic mice (FIG. 4E). Similar to Limk1^{-/-} mice, chronic treatment with Compound 1 delayed learning in non-transgenic mice. Compound 1 administration improved memory performance of APP23 mice after four months of treatment.

1. A method for treating, delaying the onset of, or ameliorating at least one symptom of, dementia associated with β-amyloid (Aβ) accumulation, the method comprising administering to a subject in need thereof an effective amount of an inhibitor of LIMK1, wherein the inhibitor comprises a compound of Formula (I) or a pharmaceutically acceptable salt thereof: 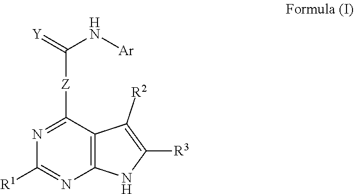
wherein:
Z is selected from the group consisting of optionally substituted cycloalkylene, optionally substituted arylene and optionally substituted aniline;
R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;
Y is selected from the group consisting of O, S, NCN, NCS and NSO₂Me; and
Ar is selected from the group consisting of optionally substituted aryl, optionally substituted heteroaryl and optionally substituted heterocyclyl.

2-14. (canceled)

15. The method according to claim 1, wherein Z is selected from one of the following structures: 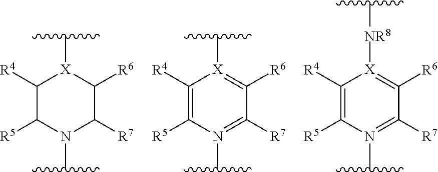
wherein:
R⁴, R⁵, R⁶ and R⁷ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl ;
X is CH or N; and
R⁸ is H or optionally substituted alkyl.

16. The method according to claim 1, wherein the compound of Formula (I) is the compound of Formula (Ia) or a pharmaceutically acceptable salt thereof: 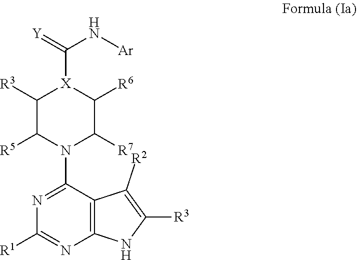
wherein variables R¹ to R⁷ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl
X is CH or N;
Y is selected from the group consisting of O, S, NCN, NCS and NSO₂Me; and
Ar is selected from the group consisting of optionally substituted aryl, optionally substituted heteroaryl and optionally substituted heterocyclyl .

17. The method according to claim 1, wherein the compound of Formula (I) is the compound of Formula (Ia), or a pharmaceutically acceptable salt thereof, wherein:
R¹, R³, R⁵, R⁶ and R⁷ are H;
R² is methyl;
R³ is (S)-methyl;
X is N;
Y is NCN; and
Ar is 3-bromophenyl.

18. The method according to claim 16, wherein the compound of Formula (Ia) has the following structure:

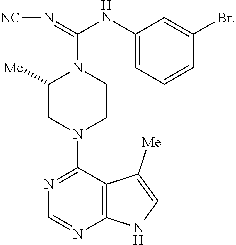 [SMILES: Cc1c[nH]c2ncnc(N3CCN(/C(=N\C#N)Nc4cccc(Br)c4)[C@@H](C)C3)c12]

19. The method according to claim 1, wherein the compound is a selective inhibitor of LIMK1.

20. The method according to claim 1, wherein the compound is a specific inhibitor of LIMK1.

21. The method according to claim 1, wherein the dementia is Alzheimer's disease.

22. The method according to claim 1, wherein the at least one symptom of the dementia comprises memory deficits and/or aberrations or disintegration of neuronal networks.

23. The method according to claim 22, wherein aberrations or disintegration of neuronal networks are associated with excitotoxicity or A13 toxicity.

24. A method for improving memory in a subject suffering from dementia associated with β-amyloid (Aβ) accumulation, the method comprising administering to a subject in need thereof an effective amount of an inhibitor of LIMK1, wherein the inhibitor comprises a compound of Formula (I) or a pharmaceutically acceptable salt thereof: 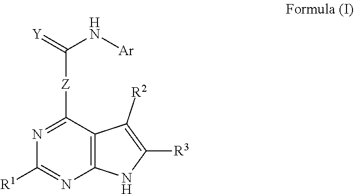
wherein:
Z is selected from the group consisting of optionally substituted cycloalkylene, optionally substituted arylene and optionally substituted aniline;
R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;
Y is selected from the group consisting of O, S, NCN, NCS and NSO₂Me; and
Ar is selected from the group consisting of optionally substituted aryl, optionally substituted heteroaryl and optionally substituted heterocyclyl.

25. A method for reducing Aβ toxicity in neurons, the method comprising exposing neurons to an effective amount of an inhibitor of LIMK1, wherein the inhibitor comprises a compound of Formula (I) or a pharmaceutically acceptable salt thereof: 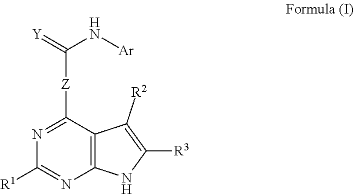
wherein:
Z is selected from the group consisting of optionally substituted cycloalkylene, optionally substituted arylene and optionally substituted aniline;
R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;
Y is selected from the group consisting of O, S, NCN, NCS and NSO₂Me; and
Ar is selected from the group consisting of optionally substituted aryl, optionally substituted heteroaryl and optionally substituted heterocyclyl.
